(19) United States
(12) Patent Application Publication
(10) Pub. No.: US 2025/0297200 A1
(43) Pub. Date: Sep. 25, 2025

(54) Kirigami Tissue Fabrication and Testing Platform

(71) Applicant: THE REGENTS OF THE UNIVERSITY OF MICHIGAN, Ann Arbor, MI (US)

(72) Inventors: Max Shtein, Ann Arbor, MI (US); Katherine Wei, Ann Arbor, MI (US); Brendon Baker, Ann Arbor, MI (US); Austin Stis, Ann Arbor, MI (US); Samuel DePalma, Ann Arbor, MI (US)

(21) Appl. No.: 18/853,033

(22) PCT Filed: Mar. 30, 2023
(86) PCT No.: PCT/US2023/016800

§ 371 Date: Sep. 30, 2024

Related U.S. Application Data
(60) Provisional application No. 63/325,495, filed on Mar. 30, 2022.

(51) Int. Cl. C12M 3/00; B23K 26/38; B23K 26/402; C12M 1/12; C12M 1/34; C12M 1/42

(52) U.S. Cl. CPC C12M 21/08; B23K 26/38; B23K 26/402; C12M 25/04; C12M 35/04; C12M 41/46

(57) ABSTRACT

Systems and methods for fabricating an adjustable width kirigami structure for tissue fabrication and testing are disclosed. An example method includes forming, by a laser cutting device, at least two kirigami structure substrates that include a plurality of bendable pillars. The example method further includes bending each of the plurality of bendable pillars to an erected position for each of the at least two kirigami structure substrates. The example method further includes layering a first kirigami structure substrate of the at least two kirigami structure substrates over a second kirigami structure substrate of the at least two kirigami structure substrates, such that the first kirigami structure substrate is antiparallel relative to the second kirigami structure substrate, to generate an adjustable width kirigami structure.

### CROSS-REFERENCE TO RELATED APPLICATIONS

This application claims priority to U.S. Provisional Patent Application No. 63/325,495, entitled “Kirigami Tissue Fabrication and Testing Platform,” filed on Mar. 30, 2022, the disclosure of which is hereby incorporated herein by reference.

### STATEMENT REGARDING FEDERALLY SPONSORED RESEARCH OR DEVELOPMENT

This invention was made with government support under 1647837, 2029139, and 2033654 awarded by the National Science Foundation. The government has certain rights in the invention.

### FIELD OF THE DISCLOSURE

The present disclosure relates generally to in vitro tissue fabrication and, more particularly, to kirigami tissue fabrication and testing platforms.

### BACKGROUND

The background description provided herein is for the purpose of generally presenting the context of the disclosure. Work of the presently named inventor, to the extent it is described in this background section, as well as aspects of the description that may not otherwise qualify as prior art at the time of filing, are neither expressly nor impliedly admitted as prior art against the present disclosure.

Generally speaking, when developing new drugs, cardiotoxicity needs to be tested. In vitro cardiomyocytes provide a viable base to test cardiotoxicity and study disease, and such testing typically requires mature cardiomyocytes. However, conventional testing platforms (also referenced herein as “structures”) configured to host and grow in vitro cardiomyocytes (e.g., induced pluripotent stem cell derived cardiomyocytes (iPSC-CMs)) suffer from several notable drawbacks that either directly impede the maturation process for cardiomyocytes or otherwise limit the practical adoption of such conventional testing platforms.

For example, studies have shown that physical and electrical stimulation aid in the maturation of in vitro cardiomyocytes. Some conventional structures can provide a certain amount of physical stimulation, but creating these conventional structures requires the fabrication of molds to cast the pillars and separate, additional molds for the support structures. Creating each of these individual components for such multiple-part platforms is a very complicated manufacturing process that is not viable for high throughput testing. Simply put, conventional testing structures suffer from a litany of challenges resulting from, among other issues, materials resulting in manufacturing difficulties, highly limited scale, and poor integration with electrical stimulation components.

Therefore, there is a need for improved in vitro tissue fabrication and testing platforms that provide mechanical and electrical stimulation to promote the formation, growth, and maturation of in vitro tissue samples in a manner that improves testing capabilities of new pharmaceuticals relative to conventional structures.

### SUMMARY OF THE INVENTION

According to an embodiment of the present disclosure, a method of fabricating an adjustable width kirigami structure for tissue fabrication and testing is disclosed. The method comprises: forming, by a laser cutting device, at least two kirigami structure substrates that include a plurality of bendable pillars; bending each of the plurality of bendable pillars to an erected position for each of the at least two kirigami structure substrates; and layering a first kirigami structure substrate of the at least two kirigami structure substrates over a second kirigami structure substrate of the at least two kirigami structure substrates, such that the first kirigami structure substrate is antiparallel relative to the second kirigami structure substrate, to generate an adjustable width kirigami structure.

In a variation of this embodiment, the method further comprises: positioning the adjustable width kirigami structure in a well plate for maturation of in vitro tissues. Further in this variation, the method further comprises: positioning the adjustable width kirigami structure in the well plate, wherein the well plate has a plurality of rectangular wells that are each configured to receive a pair of pillars from the plurality of bendable pillars, and wherein each pair of pillars includes (i) a first pillar from the plurality of bendable pillars included on the first kirigami structure substrate and (ii) a second pillar from the plurality of bendable pillars included on the second kirigami structure substrate. Yet further in this variation, the method further comprises: positioning the adjustable width kirigami structure in the well plate, wherein the well plate has a plurality of wells that are configured to form the in vitro tissues with a 2:1 aspect ratio.

In another variation of this embodiment, the method further comprises: forming, by the laser cutting device, the at least two kirigami structure substrates that include the plurality of bendable pillars, wherein each kirigami structure substrate of the at least two kirigami structure substrates includes an electrode inlay configured to provide electrical stimulation to an in vitro tissue in contact with at least one pillar of the plurality of bendable pillars.

In yet another variation of this embodiment, the method further comprises: forming, by the laser cutting device, the at least two kirigami structure substrates that include a plurality of bendable pillars, wherein the at least two kirigami structure substrates are comprised of at least one of (i) polyethylene terephthalate (PET) or (ii) polyimide.

In still another variation of this embodiment, the method further comprises: bending, by thermoforming, each of the plurality of bendable pillars to the erected position for each of the at least two kirigami structure substrates.

In yet another variation of this embodiment, the method further comprises: layering the first kirigami structure substrate of the at least two kirigami structure substrates over the second kirigami structure substrate of the at least two kirigami structure substrates, such that the first kirigami structure substrate is antiparallel relative to the second kirigami structure substrate, to generate the adjustable width kirigami structure, wherein each pillar of the first kirigami structure forms a respective pair with a corresponding pillar from the second kirigami structure, and each respective pair is offset by about 5 millimeters (mm).

In still another variation of this embodiment, the method further comprises: causing an in vitro tissue to span a width between adjacent pillars of the adjustable width kirigami structure, wherein the adjacent pillars are positioned in a well of a well plate.

In yet another variation of this embodiment, the method further comprises: providing, by the adjustable width kirigami structure, mechanical stimulation to an in vitro tissue to promote maturation of the in vitro tissue.

According to another embodiment of the present disclosure, a system for tissue fabrication and testing is disclosed. The system comprises: an adjustable width kirigami structure, comprising: a first kirigami structure substrate that includes a first plurality of bendable pillars that are bent into an erected position, and a second kirigami structure substrate that includes a second plurality of bendable pillars that are bent into the erected position, wherein the first kirigami structure substrate is layered over the second kirigami structure substrate, such that the first kirigami structure substrate is antiparallel relative to the second kirigami structure substrate; and a well plate including a plurality of wells configured to receive pillars from (i) the first plurality of bendable pillars and (ii) the second plurality of bendable pillars.

In a variation of this embodiment, each pillar of the first kirigami structure forms a respective pair with a corresponding pillar from the second kirigami structure, and the wells are configured to receive the respective pairs from adjustable width kirigami structure.

In another variation of this embodiment, the first kirigami structure substrate and the second kirigami structure substrate are formed using a laser cutting device.

In yet another variation of this embodiment, the first plurality of bendable pillars and the second plurality of bendable pillars are bent into the erected position by a thermoforming device, and wherein each pillar of the first plurality of bendable pillars and the second plurality of bendable pillars has (i) a rectangular shape, (ii) a width value of about 2.5 millimeters, and (iii) a thickness value of between about 20 micrometers (μm) and 50 μm.

In still another variation of this embodiment, each well in the plurality of wells has a rectangular shape configured to form in vitro tissues with a 2:1 aspect ratio.

In yet another variation of this embodiment, the first kirigami structure substrate and the second kirigami structure substrate both include an electrode inlay configured to provide electrical stimulation to an in vitro tissue in contact with at least one pillar of the plurality of bendable pillars.

In still another variation of this embodiment, the adjustable width kirigami structure is comprised of at least one of (i) polyethylene terephthalate (PET) or (ii) polyimide.

In yet another variation of this embodiment, the first kirigami structure substrate is layered over the second kirigami structure substrate, such that the first kirigami structure substrate is antiparallel relative to the second kirigami structure substrate, each pillar of the first kirigami structure forms a respective pair with a corresponding pillar from the second kirigami structure, and each respective pair is offset by about 5 millimeters (mm).

In still another variation of this embodiment, an in vitro tissue spans a width between adjacent pillars of the adjustable width kirigami structure, wherein the adjacent pillars are positioned in a respective well of the well plate.

In yet another variation of this embodiment, each pillar of the first plurality of bendable pillars and the second plurality of bendable pillars has a relief or a pattern configured to increase a strength of adhesion of an in vitro tissue to each pillar.

### BRIEF DESCRIPTION OF THE DRAWINGS

The figures described below depict various aspects of the system and methods disclosed herein. It should be understood that each figure depicts an embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

FIG. 1 illustrates an example system workflow for fabrication of an adjustable width kirigami structure that can be combined with a well plate to form a tissue fabrication and testing system, in accordance various aspects disclosed herein.

FIGS. 2A-2E illustrate fabrication of an adjustable width kirigami structure, in accordance various aspects disclosed herein.

FIGS. 3A-3D illustrate combining the adjustable width kirigami structure with a well plate to form a tissue fabrication and testing system that is configured to grow/mature tissue samples, in accordance with various aspects disclosed herein.

FIG. 4 illustrates example locations of mechanical/physical stimulation provided by pillars of the adjustable width kirigami structure to an in vitro tissue sample within a well of the tissue fabrication and testing system, in accordance with various aspects disclosed herein.

FIG. 5 illustrates an example method for fabrication of an adjustable width kirigami structure, in accordance various aspects disclosed herein.

### DETAILED DESCRIPTION

As previously mentioned, the kirigami tissue fabrication and testing platforms described herein generally include a substrate and well plate combination configured to form, mature, and monitor tissues in vitro. The substrate includes pairs of pillars that are registered with the well plate, such that each well receives a precisely positioned pair of pillars from the substrate. Substrates and resulting pillars are easily customized in terms of geometry and mechanics by laser cutting the starting sheet, such that any suitable size/geometry of pillar is easily achievable. The well plate is designed to have wells, which may be, for example, rectangular in shape, to promote the assembly of elongated tissues spanning between the pairs of pillars that are positioned in the wells.

As a result of this platform configuration, the present disclosure includes improvements to other technologies or technical fields at least because the present disclosure describes or introduces improvements in the field of drug development and compound screening. More specifically, the kirigami tissue fabrication and testing platforms of the present disclosure directly advance/improve the fields of drug development and compound screening. The kirigami tissue fabrication and testing platforms of the present disclosure enable straightforward and high throughput generation of 3D microtissues (also referenced herein as “tissues”) that can be used as physiologic models for compound screening. The kirigami tissue fabrication and testing platforms also readily incorporate mechanical, electrical, and/or chemical stimulation for promoting the growth and maturation of tissues in vitro. Overall, the kirigami tissue fabrication and testing platforms constitute a foundation for a low-cost test platform that improves over conventional techniques at least because such conventional techniques lack the ability to easily adapt to and scale with industry-standard multi-well assays and medium/high throughput testing protocols and hardware. As a result, the platforms of the present disclosure reduce the cost and accelerate the testing of potential therapies (e.g., cardiotoxicity assessment).

In addition, the present disclosure includes specific features other than what is well-understood, routine, conventional activity in the field, or adding unconventional steps that confine the claim to a particular useful application, e.g., layering a first kirigami structure substrate of the at least two kirigami structure substrates over a second kirigami structure substrate of the at least two kirigami structure substrates, such that the first kirigami structure substrate is antiparallel relative to the second kirigami structure substrate, to generate an adjustable width kirigami structure. In this manner, the techniques of the present disclosure provide a means of reducing the spacing between the facing elements (e.g., pillars) of the kirigami structure relative to the height of the facing elements that is not well-understood, routine, or conventional activity in the field of drug development and compound screening.

To provide a general understanding of the system(s)/components utilized in the techniques of the present disclosure, FIG. 1 illustrates an example system workflow 100 that is configured for fabrication of an adjustable width kirigami structure that can be combined with a well plate to form a tissue fabrication and testing system. It should be appreciated that the example system workflow 100 is merely an example and that alternative or additional components are envisioned.

As illustrated in FIG. 1, the example system workflow 100 includes a substrate 102, a fabrication component 104, an adjustable width kirigami structure 106, a well plate 108, and a tissue fabrication and testing system 110. The fabrication component 104 may include a laser cutting device 104a and a thermoforming device 104b. Generally speaking, the fabrication component 104 may receive the substrate 102, and may generate the adjustable width kirigami structure 106. Thereafter, the adjustable width kirigami structure 106 may be combined with the well plate 108 to generate the tissue fabrication and testing system 110.

More specifically, the substrate 102 may be a material that the fabrication component 104 can cut, bend, and/or otherwise adjust into a kirigami substrate structure that includes bendable pillars for forming into the adjustable width kirigami structure 106. For example, in certain embodiments, the substrate 102 may be comprised of at least one of (i) polyethylene terephthalate (PET), (ii) polyimide, and/or any other suitable material.

The fabrication component 104 may generally include suitable components/devices in order to fabricate the adjustable width kirigami structure 106 from the substrate 102. The laser cutting device 104a may include any device capable of cutting the substrate 102 to include the pillars that are positioned inside wells of the well plate 108 and thereby provide mechanical and electric stimulation to an in vitro tissue sample. The thermoforming device 104b may include any device capable of bending and/or otherwise forming the pillars cut into the substrate 102 into an erected position relative to the substrate, such that the pillars may be positioned into wells of the well plate 108 when the adjustable width kirigami structure 106 is combined with the well plate 108. Of course, the laser cutting device 104a may cut the substrate with a laser and the thermoforming device 104b may bend the pillars into the erected position by thermoforming the pillars into the erected position, but it should be understood that cutting and bending the pillars of the substrate 102 may be performed by any suitable devices. As an example, cutting the substrate 102 may also be performed by a stamping tool, a die cutting tool, and/or any other suitable device or combinations thereof.

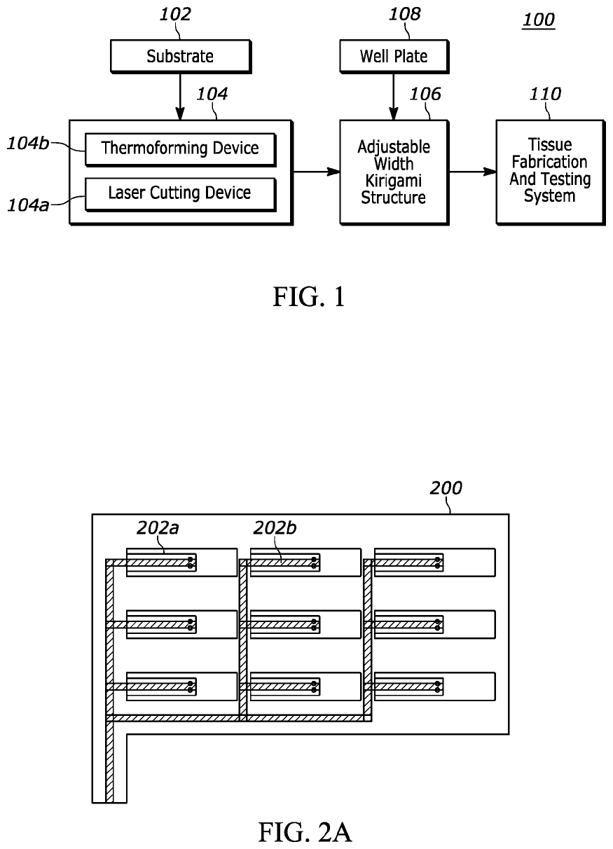

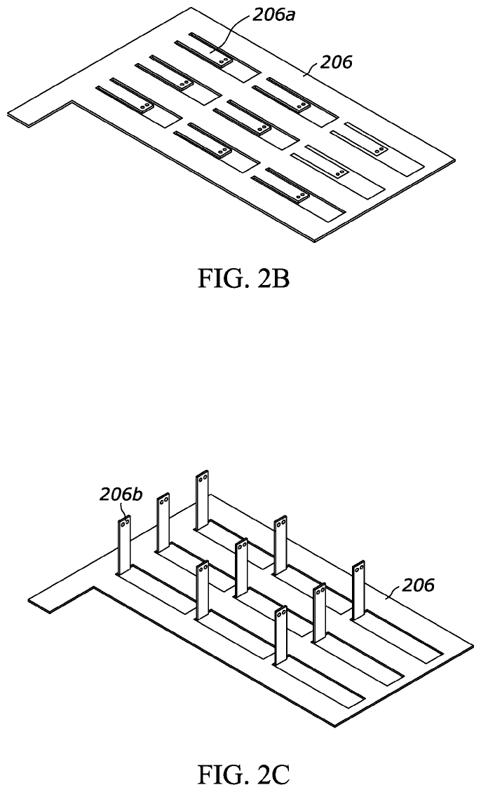

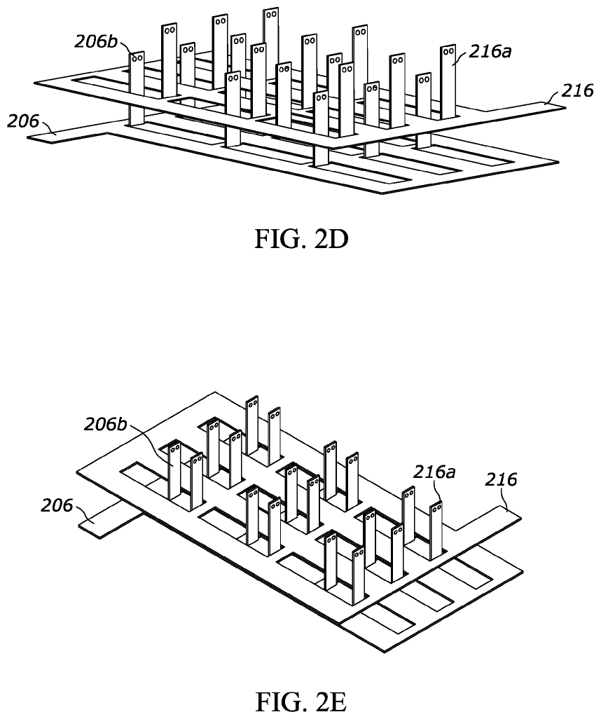

As a result of the fabrication performed on the substrate 102 by the fabrication component 104, the substrate 102 may be formed into two or more kirigami structure substrates that may be combined into the adjustable width kirigami structure 106. Generally, the adjustable width kirigami structure 106 is comprised of two layered kirigami structure substrates such that a first kirigami structure substrate is antiparallel relative to a second kirigami structure substrate. As referenced herein, a first kirigami structure substrate is “antiparallel” relative to a second kirigami structure substrate when the first kirigami structure substrate is rotated by 180° relative to the second kirigami structure substrate, such that the two kirigami structure substrates may be layered together in a manner that creates pillar pairs extending in a single direction having a width/spacing between the two pillars of each pillar pair (e.g., as illustrated in FIGS. 2D and 2E). Moreover, the width/spacing between pillars of the adjustable width kirigami structure 106 forming pairs within wells of the well plate 108 may be adjusted, such that the pillars may more optimally occupy space within wells of the well plate 108, and/or to more optimally stimulate growth and maturation of a tissue sample of a desired size.

In any event, the tissue fabrication and testing system 110 is formed by combining the adjustable width kirigami structure 106 and the well plate 108. More specifically, and as previously mentioned, the pillars of the adjustable width kirigami structure 106 may be positioned within wells of the well plate 108, such that in vitro tissues may be grown and matured inside the wells and may receive mechanical and electrical stimulation from the pillar pairs positioned within the wells. Thus, due in part to the unique fabrication processes of the adjustable width kirigami structure 106, the tissue fabrication and testing system 110 effectively fabricates in vitro tissues in a manner that is consistent, easily repeatable, and scalable for high throughput testing.

FIGS. 2A-2E illustrate fabrication of an adjustable width kirigami structure, in accordance various aspects disclosed herein. In particular, FIG. 2A illustrates a unit cell 200 of a kirigami substrate pattern, which includes pattern outlines representative of pillars 202a and electrodes 202b that may be included as part of a kirigami structure substrate (e.g., used to form the adjustable width kirigami structure 106). As illustrated in FIG. 2A the pillars 202a may be positioned on the unit cell 200 to form several rows/columns of adjacent pillars 202a. The electrodes 202b may be inlaid/deposited in the material of the unit cell 200, and the electrodes 202b may extend through the entire length of each pillar 202a (as shown in FIG. 2A) in order to apply electrical stimulus through the substrate to the in vitro tissues. Additionally, the electrodes 202b may be connected to traces (not shown) that are configured to facilitate the addressing of the electrodes 202b (e.g., all in parallel, in a multiplexed fashion, etc.). However, it should be understood that the substrate structures used to form the adjustable width kirigami structure 106 may include pillars 202a of any suitable length/geometry, and may include electrodes 202b that extend any suitable length along the pillars 202a, including no electrodes 202b.

FIG. 2B illustrates a single layer structure substrate 206 (also referenced herein as a “first single layer structure substrate 206”) that has been cut to include multiple pillars 206a. Such cutting may be performed by, for example, the laser cutting device 104a of FIG. 1. The cut-out sections of the single layer structure substrate 206 may enable adjustments to the width/spacing between pillar pairs of the resulting adjustable width kirigami structure 106. When the pillars 206a are successfully cut and/or otherwise formed on the single layer structure substrate 206, the single layer structure substrate 206 may be thermoformed to position the pillars 206a into an erected position, as illustrated in FIG. 2C. In particular, the thermoforming device 104b may receive the single layer structure substrate 206 after the pillars 206a are cut into the single layer structure substrate 206, and the thermoforming device 104b may thermoform each of the pillars 206a into the erected pillars 206b illustrated in FIG. 2C.

When two single layer structure substrates 206, 216 have been cut by the laser cutting device 104a and have the pillars thermoformed into the erected pillars 206b, 216a by the thermoforming device 104b, the two structure substrates 206, 216 may be combined in a manner similar to that shown in FIG. 2D. The first single layer structure substrate 206 and the second single layer structure substrate 216 may be layered together, such that the second single layer structure substrate 216 is placed over the first single layer structure substrate 206. More specifically, the second single layer structure substrate 216 may be layered in an antiparallel direction over the first single layer structure substrate 206, and in this manner, the erected pillars 216a of the second single layer structure substrate 216 may form spaced pairs with the erected pillars 206b of the first single layer structure substrate 206.

For a clearer illustration, FIG. 2E provides a slightly overhead perspective of the layered structure substrates 206, 216. As illustrated in FIG. 2E, each erected pillar 206b has an adjacent erected pillar 216a, such that the erected pillars 206b, 216a of the single layer structure substrates 206, 216 form a plurality of pairs of erected pillars that are configured to stimulate growth and maturation of in vitro tissues when positioned in wells of a well plate (e.g., well plate 108). Accordingly, when the single layer structure substrates 206, 216 are layered in the configuration illustrated in FIG. 2E, and the spacing/width between the pillar pairs reaches a desirable distance, the substrates 206, 216 may form the adjustable width kirigami structure (e.g., adjustable width kirigami structure 106). In certain embodiments, forming the adjustable width kirigami structure may also include bonding the single layer structure substrates 206, 216 together in any suitable fashion when the spacing/width between the pillar pairs reaches a desirable distance.

Moreover, it should be appreciated that the adjustable width kirigami structure may be or include any suitable number of substrate layers that each include any suitable number of pillars to create any suitable number of pillar pairs for insertion into any suitable number of wells in a well plate. For example, a first adjustable width kirigami structure may include four substrate layers that each include 48 individual pillars. A first pair and a second pair of the substrate layers may be layered similar to the layered structure substrates 206, 216, such that the two pairs of substrate layers create 96 individual pillar pairs for insertion into a well plate with 96 individual wells. In another example, and as illustrated in FIGS. 2A-2E, a second adjustable width kirigami structure may include two substrate layers that each include nine individual pillars. The two substrate layers may be layered similar to the layered structure substrates 206, 216, such that the pair of substrate layers create nine individual pillar pairs for insertion into a well plate with nine individual wells.

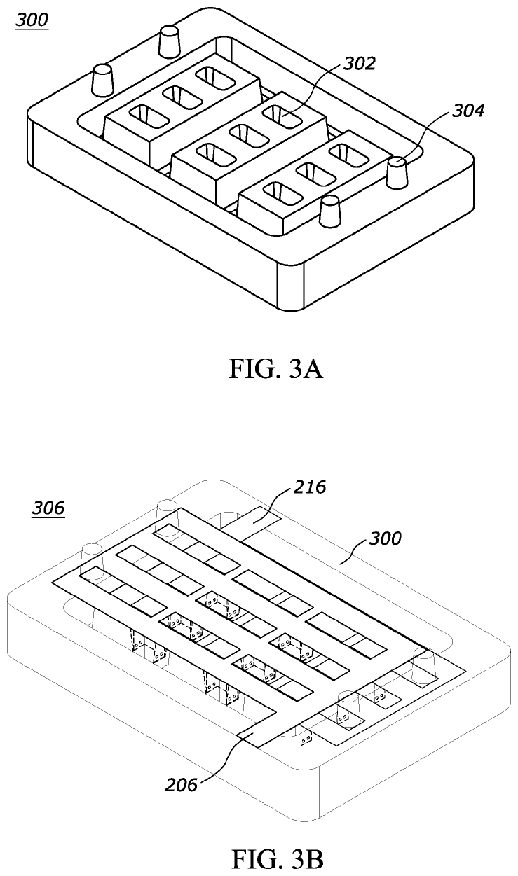

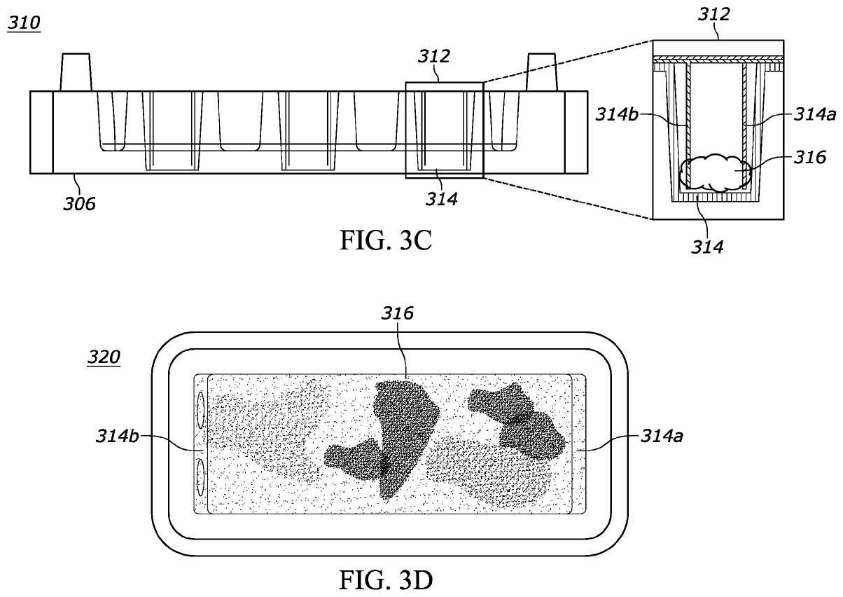

In any event, when the adjustable width kirigami structure 106 is formed, the structure 106 may be positioned in/on a well plate (e.g., well plate 108) to complete the construction of a tissue fabrication and testing system (e.g., tissue fabrication and testing system 110). FIGS. 3A-3D illustrate combining the adjustable width kirigami structure with a well plate to form a tissue fabrication and testing system that is configured to grow/mature tissue samples, in accordance with various aspects disclosed herein.

FIG. 3A illustrates an exemplary well plate 300 that may be combined with an adjustable width kirigami structure. The exemplary well plate 300 includes a plurality of wells 302 that are each configured to receive a pair of pillars from adjustable width kirigami structure. The wells 302 may be, for example, rectangular in shape and/or any other suitable shape(s) (e.g., round) or combinations thereof, and may be of any suitable depth in order to receive the pillar pairs.

In general, the well plate 300 is configured to have thin glass bottoms for each individual well 302 to allow for easy imaging of live or fixed tissue samples using inverted microscopes. More specifically, the bottom of the wells 302 may be made from glass cover slides that have a high transmittance and enable imaging with objectives that have a relatively short working distance. Further, the well plate 300 may receive a chemical surface modification in order for tissues to assemble within the individual wells 302 of the well plate 300. In particular, well plate 300 may be treated with pluronics and/or any other suitable non-fowling coating or combinations thereof to prevent the tissues from adhering to the well 302 walls. In this manner, the coating applied to the well 302 walls forces the tissues to compact around the pillars and generate a well-formed tissue. In certain embodiments, the wells 302 may be configured to shape the in vitro tissue samples to a height-to-width aspect ratio of 2:1.

Further, the exemplary well plate 300 includes multiple attachment pegs 304 that are configured to receive slotted portions of the adjustable width kirigami structure in order to keep the structure in place on the well plate 300. This positioning of the pillar pairs and slotted sections of the adjustable width kirigami structure with the exemplary well plate 300 is illustrated more clearly in FIG. 3B. As shown in FIG. 3B, the single layer structure substrates 206, 216 comprising the adjustable width kirigami structure are inverted and positioned on the exemplary well plate 300 so that the pillar pairs are inserted into the wells 302 and the slotted portions of the structure receive the attachment pegs 304, thereby forming the tissue fabrication and testing system 306.

FIG. 3C illustrates a side view 310 of the tissue fabrication and testing system 306 including a detailed side view 312 of a particular well 314. As illustrated in FIG. 3C, the particular well 314 includes two pillars 314a, 314b that are positioned in the particular well 314, as well as an in vitro tissue 316 that is growing/maturing between the two pillars 314a, 314b. As a result of the two pillars 314a, 314b mechanically and electrically stimulating the in vitro tissue 316, the tissue 316 may mature between the two pillars 314a, 314b in a manner sufficient for testing (e.g., cardiotoxicity testing). This maturation is additionally illustrated in the top view 320 of FIG. 3D, which is a rendering of an exemplary optical micrograph of the in vitro tissue 316 spanning between the two pillars 314a, 314b of the particular well 314.

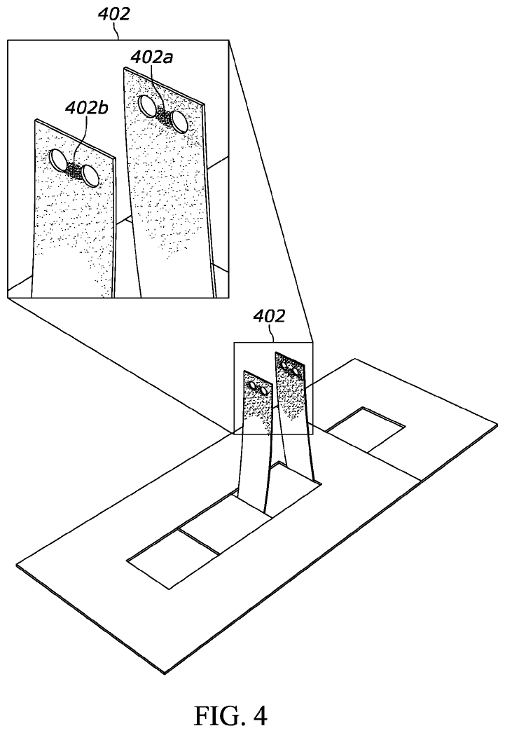

FIG. 4 illustrates example locations of mechanical/physical stimulation provided by pillars of the adjustable width kirigami structure to an in vitro tissue sample within a well of the tissue fabrication and testing system, in accordance with various aspects disclosed herein. Namely, the stress diagram 400 of FIG. 4 highlights stress locations 402 near the ends of two exemplary pillars. The stress locations 402 include a first high stress location 402a on a first pillar, and a second high stress location 402b on a second pillar. These high stress locations 402a, 420b represent areas of the respective pillars where, for example, the most mechanical stress is exerted on/by the respective pillars from/to the in vitro tissue growing/maturing between the two pillars (e.g., as shown in FIGS. 3C and 3D). This mechanical stress is also indicative of the mechanical stimulation provided by the two pillars while the in vitro tissue is growing and maturing. For example, maturation of the in vitro tissue is aided by allowing the tissues to contract against the mechanical resistance provided by the pillars. Thus, the pillar thickness, the pillar geometry, the pillar length, the pillar width, the pillar material composition, and/or other aspects of the pillar geometry can be selected for optimal compatibility (e.g., mechanical resistance characteristics) with what each particular tissue requires. As an example, rounded pillars, “dog-bone” shaped pillars, and/or pillars with other geometries may be designed to optimally alter the mechanics of tissue formation within the wells in certain circumstances.

However, it should be understood that these high stress locations 402a, 402b are for the purposes of illustration only, and that the pillars may provide mechanical stimulation to the in vitro tissue from any suitable contact location. Moreover, the high stress locations 402a, 402b may also correspond to locations of the respective pillars where the embedded electrodes provide electrical stimulation to the in vitro tissue. Of course, as previously discussed in reference to FIG. 2A, the electrodes may extend along the entire length of the respective pillars, and the high stress locations 402a, 402b may represent the lateral locations on the surfaces of the pillars contacting the in vitro tissue where the electrical stimulation may originate. However, it should be appreciated that the electrodes may be embedded/inlaid at any suitable location within/on the pillars, and as a result, the electrodes may provide electrical stimulation originating from any location of the pillars contacting the in vitro tissue.

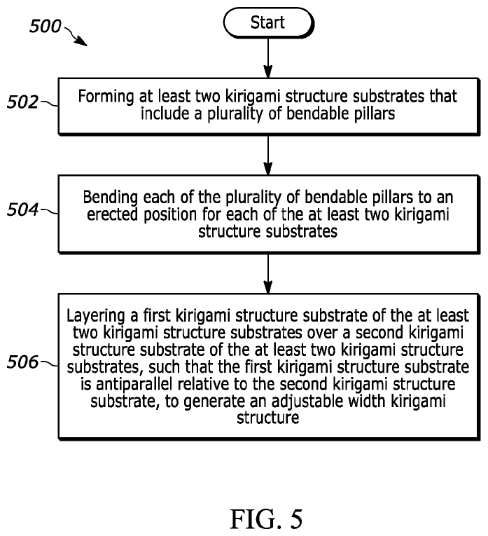

FIG. 5 illustrates an example method 500 for fabrication of an adjustable width kirigami structure (e.g., adjustable width kirigami structure 106), in accordance various aspects disclosed herein. For ease of discussion, many of the various actions included in the method 500 may be optional, and the various actions included in the method 500 may be performed by, for example, the thermoforming device 104a, the laser cutting device 104b, and/or any suitable components or combinations thereof.

The method 500 includes forming at least two kirigami structure substrates (e.g., single layer structure substrate 206) that include a plurality of bendable pillars (block 502). In certain embodiments, the method 500 further includes forming, by the laser cutting device (e.g., laser cutting device 104a), the at least two kirigami structure substrates that include the plurality of bendable pillars, wherein each kirigami structure substrate of the at least two kirigami structure substrates includes an electrode inlay configured to provide electrical stimulation to an in vitro tissue in contact with at least one pillar of the plurality of bendable pillars. Such an electrode inlay may be inlaid into the kirigami structure substrates through thin film deposition by vacuum thermal evaporation or electron beam deposition, electro/less plating, and/or any other suitable method or combinations thereof.

In some embodiments, the method 500 further includes forming, by the laser cutting device 104a, the at least two kirigami structure substrates that include a plurality of bendable pillars, wherein the at least two kirigami structure substrates are comprised of at least one of (i) polyethylene terephthalate (PET) or (ii) polyimide.

The method 500 further includes bending each of the plurality of bendable pillars to an erected position for each of the at least two kirigami structure substrates (block 504). In some embodiments the method 500 further includes bending, by thermoforming, each of the plurality of bendable pillars to the erected position for each of the at least two kirigami structure substrates. This thermoforming may be performed by, for example, the thermoforming device 104b.

Further, in certain embodiments, each pillar of the plurality of bendable pillars has a relief or a pattern configured to increase a strength of adhesion of an in vitro tissue to each pillar. For example, each pillar illustrated in FIGS. 2A-2E (e.g., pillars 202a, 206a, 206b, 216a) and FIG. 4 includes two holes near the top of the pillars which are configured to increase the strength of adhesion of the in vitro tissue to the pillars.

The method 500 further includes layering a first kirigami structure substrate of the at least two kirigami structure substrates over a second kirigami structure substrate of the at least two kirigami structure substrates, such that the first kirigami structure substrate is antiparallel relative to the second kirigami structure substrate, to generate an adjustable width kirigami structure (block 506).

In certain embodiments, the method 500 further includes positioning the adjustable width kirigami structure in a well plate for maturation of in vitro tissues. Further in these embodiments, the method 500 may include positioning the adjustable width kirigami structure in the well plate, wherein the well plate has a plurality of rectangular wells that are each configured to receive a pair of pillars from the plurality of bendable pillars, and wherein each pair of pillars includes (i) a first pillar from the plurality of bendable pillars included on the first kirigami structure substrate and (ii) a second pillar from the plurality of bendable pillars included on the second kirigami structure substrate. Additionally, or alternatively, these embodiments may include positioning the adjustable width kirigami structure in the well plate, wherein the well plate has a plurality of wells that are configured to form the in vitro tissues with a 2:1 aspect ratio. As previously discussed, the 2:1 aspect ratio may reference a ratio of the height-to-width of the in vitro tissue and/or any other suitable dimensions or combinations thereof. Of course, the aspect ratio of the in vitro tissue may be any suitable ratio (e.g., 2:1, 3:1, 3:2, etc.) describing any suitable combination of dimensions.

In some embodiments, the method 500 may further include layering the first kirigami structure substrate of the at least two kirigami structure substrates over the second kirigami structure substrate of the at least two kirigami structure substrates, such that the first kirigami structure substrate is antiparallel relative to the second kirigami structure substrate, to generate the adjustable width kirigami structure. In these embodiments, each pillar of the first kirigami structure forms a respective pair with a corresponding pillar from the second kirigami structure, and each respective pair is offset by about 5 millimeters (mm).

In certain embodiment, the method 500 may further include causing an in vitro tissue to span a width between adjacent pillars of the adjustable width kirigami structure, wherein the adjacent pillars are positioned in a well of a well plate. Moreover, in some embodiments, the method 500 may further include providing, by the adjustable width kirigami structure, mechanical stimulation to an in vitro tissue to promote maturation of the in vitro tissue. Such mechanical stimulation may be the result of the particular mechanical characteristics of the individual pillars (e.g., thickness, material composition, length, etc.), as well as the movement of the individual pillars relative to one another from pressure applied by the tissues that may result in increases/decreases to the width/spacing between pillar pairs (e.g., movements of the layered kirigami structure substrates).

### Additional Considerations

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connects the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of the example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but also deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but also deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

While the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

The foregoing description is given for clearness of understanding; and no unnecessary limitations should be understood therefrom, as modifications within the scope of the invention may be apparent to those having ordinary skill in the art.

What is claimed:

1. A method of fabricating an adjustable width kirigami structure for tissue fabrication and testing, comprising:
forming, by a laser cutting device, at least two kirigami structure substrates that include a plurality of bendable pillars;
bending each of the plurality of bendable pillars to an erected position for each of the at least two kirigami structure substrates; and
layering a first kirigami structure substrate of the at least two kirigami structure substrates over a second kirigami structure substrate of the at least two kirigami structure substrates, such that the first kirigami structure substrate is antiparallel relative to the second kirigami structure substrate, to generate an adjustable width kirigami structure.

2. The method of claim 1, further comprising:
positioning the adjustable width kirigami structure in a well plate for maturation of in vitro tissues.

3. The method of claim 2, further comprising:
positioning the adjustable width kirigami structure in the well plate, wherein the well plate has a plurality of rectangular wells that are each configured to receive a pair of pillars from the plurality of bendable pillars, and wherein each pair of pillars includes (i) a first pillar from the plurality of bendable pillars included on the first kirigami structure substrate and (ii) a second pillar from the plurality of bendable pillars included on the second kirigami structure substrate.

4. The method of claim 2, further comprising:
positioning the adjustable width kirigami structure in the well plate, wherein the well plate has a plurality of wells that are configured to form the in vitro tissues with a 2:1 aspect ratio.

5. The method of claim 1, further comprising:
forming, by the laser cutting device, the at least two kirigami structure substrates that include the plurality of bendable pillars, wherein each kirigami structure substrate of the at least two kirigami structure substrates includes an electrode inlay configured to provide electrical stimulation to an in vitro tissue in contact with at least one pillar of the plurality of bendable pillars.

6. The method of claim 1, further comprising:
forming, by the laser cutting device, the at least two kirigami structure substrates that include a plurality of bendable pillars, wherein the at least two kirigami structure substrates are comprised of at least one of (i) polyethylene terephthalate (PET) or (ii) polyimide.

7. The method of claim 1, further comprising:
bending, by thermoforming, each of the plurality of bendable pillars to the erected position for each of the at least two kirigami structure substrates.

8. The method of claim 1, further comprising:
layering the first kirigami structure substrate of the at least two kirigami structure substrates over the second kirigami structure substrate of the at least two kirigami structure substrates, such that the first kirigami structure substrate is antiparallel relative to the second kirigami structure substrate, to generate the adjustable width kirigami structure,
wherein each pillar of the first kirigami structure forms a respective pair with a corresponding pillar from the second kirigami structure, and each respective pair is offset by about 5 millimeters (mm).

9. The method of claim 1, further comprising:
causing an in vitro tissue to span a width between adjacent pillars of the adjustable width kirigami structure, wherein the adjacent pillars are positioned in a well of a well plate.

10. The method of claim 1, further comprising:
providing, by the adjustable width kirigami structure, mechanical stimulation to an in vitro tissue to promote maturation of the in vitro tissue.

11. A system for tissue fabrication and testing, comprising:
an adjustable width kirigami structure, comprising:
a first kirigami structure substrate that includes a first plurality of bendable pillars that are bent into an erected position, and
a second kirigami structure substrate that includes a second plurality of bendable pillars that are bent into the erected position,
wherein the first kirigami structure substrate is layered over the second kirigami structure substrate, such that the first kirigami structure substrate is antiparallel relative to the second kirigami structure substrate; and
a well plate including a plurality of wells configured to receive pillars from (i) the first plurality of bendable pillars and (ii) the second plurality of bendable pillars.

12. The system of claim 11, wherein each pillar of the first kirigami structure forms a respective pair with a corresponding pillar from the second kirigami structure, and the wells are configured to receive the respective pairs from adjustable width kirigami structure.

13. The system of claim 11, wherein the first kirigami structure substrate and the second kirigami structure substrate are formed using a laser cutting device.

14. The system of claim 11, wherein the first plurality of bendable pillars and the second plurality of bendable pillars are bent into the erected position by a thermoforming device, and wherein each pillar of the first plurality of bendable pillars and the second plurality of bendable pillars has (i) a rectangular shape, (ii) a width value of about 2.5 millimeters, and (iii) a thickness value of between about 20 micrometers (μm) and 50 μm.

15. The system of claim 11, wherein each well in the plurality of wells has a rectangular shape configured to form in vitro tissues with a 2:1 aspect ratio.

16. The system of claim 11, wherein the first kirigami structure substrate and the second kirigami structure substrate both include an electrode inlay configured to provide electrical stimulation to an in vitro tissue in contact with at least one pillar of the plurality of bendable pillars.

17. The system of claim 11, wherein the adjustable width kirigami structure is comprised of at least one of (i) polyethylene terephthalate (PET) or (ii) polyimide.

18. The system of claim 11, wherein the first kirigami structure substrate is layered over the second kirigami structure substrate, such that the first kirigami structure substrate is antiparallel relative to the second kirigami structure substrate, each pillar of the first kirigami structure forms a respective pair with a corresponding pillar from the second kirigami structure, and each respective pair is offset by about 5 millimeters (mm).

19. The system of claim 11, wherein an in vitro tissue spans a width between adjacent pillars of the adjustable width kirigami structure, wherein the adjacent pillars are positioned in a respective well of the well plate.

20. The system of claim 11, wherein each pillar of the first plurality of bendable pillars and the second plurality of bendable pillars has a relief or a pattern configured to increase a strength of adhesion of an in vitro tissue to each pillar.
